(19) United States
(12) Patent Application Publication
(10) Pub. No.: US 2023/0157322 A1
(43) Pub. Date: May 25, 2023

(54) Keto creamy carbonated beverage formulations

(71) Applicant: Rebecca Bauman, Ft, Pierce, FL (US)

(72) Inventor: Rebecca Bauman, Ft, Pierce, FL (US)

(21) Appl. No.: 17/300,825

(22) Filed: Nov. 22, 2021

(51) Int. Cl. A23L 2/38; A23L 2/54; A23L 2/66; A23F 5/24; A23F 5/46; A23L 33/17; A23L 33/115; A23L 33/15; A23L 2/56; A23L 33/105; C12G 3/055; C12G 3/06; A61K 45/06; A61K 31/205; A61K 9/00; A61K 31/122; A61K 31/522

(52) U.S. Cl. CPC A23L 2/38; A23L 2/54; A23L 2/66; A23F 5/243; A23F 5/465; A23L 33/17; A23L 33/115; A23L 33/15; A23L 2/56; A23L 33/105; C12G 3/055; C12G 3/06; A61K 45/06; A61K 31/205; A61K 9/0095; A61K 31/122; A61K 31/522; A23V 2002/00

(57) ABSTRACT

The present invention relates to an alcoholic beverage and a non-alcoholic beverage composition intended for individuals on a Keto diet comprising about 10-25 grams of fat, about 3 grams or less of carbohydrates, about 5 grams or less of protein, 1-2 ounces of a high fat dairy cream, 5-11 ounces of carbonated water, 5 grams or less of medium-chain triglycerides (MCT), about 250 milligrams or less of a carnitine, and wherein the composition is per 12 ounce serving.

The present invention further relates to methods for producing a Keto beverage composition comprising the steps of mixing the beverage formulation ingredients, carbonating the beverage formulation ingredients, and homogenizing and/or pasteurizing the beverage formulation ingredients, wherein said beverage formulation ingredients comprise about 3 grams or less of carbohydrates, about 10-25 grams of fat, about 5 grams or less of protein, about 5 grams or less of medium-chain triglycerides (MCT), about 250 milligrams or less of a carnitine, and about 5 grams or less of a creatine, and wherein the composition is per 12 ounce serving.

### FIELD OF THE INVENTION

The present invention relates to beverages. More specifically, the present invention relates to a Keto alcoholic and a Keto non-alcoholic beverage.

### BACKGROUND OF THE INVENTION

There are three primary macronutrients in foods and drinks-carbohydrates, protein, and fats. Of these macronutrients, carbohydrates are the primary source of fuel for the central nervous system, organs and working muscles. Fat can also be used as a source of fuel for organs and working muscles but not for the central nervous system because fat cannot pass through the blood brain barrier.

On the ketogenic lifestyle, restricting carbohydrates to a range of about 15-30 grams causes the central nervous system to go into a starvation situation. The Dietary Guidelines for Americans recommends 225-325 grams of carbohydrates a day based on a 2000 calorie diet. In order to survive with such carbohydrate restriction, the brain signals to the liver to convert fats into ketones also known as ketone bodies. These ketones can pass through the blood brain barrier. There is an adaptation time frame in which the brain creates enzymes to be able to utilize ketones as an alternative primary source of fuel to carbohydrates.

A ketogenic lifestyle also requires that protein is reduced in the diet to about 80 grams or less which is far less restrictive than the carbohydrates. The reason for this restriction is that when there is excess protein, the body converts protein into glucose (carbohydrates) through a process called gluconeogenesis. These additional carbohydrates may interfere with the production of ketones and may prevent the body from using ketones as a primary fuel source. The Keto Diet does not restrict calories which in part contributes to its worldwide popularity. A diet which is very high in fat, very low in carbohydrates, 30 grams or less per day, and low in protein, 80 grams or less per day, will result in ketone production as a primary source of fuel for the central nervous system, organs, and working muscles. This metabolic state is called ketosis. Maintaining a fitness routine helps to ensure that people on a keto lifestyle stay in ketosis because if extra carbohydrates have been consumed (over 30 grams in a day) or extra protein (over 80 grams in a day) the increased activity levels will cause the working muscles to consume those additional carbohydrates ensuring ketones remain the body's primary fuel source.

Why do people decide to embrace a keto lifestyle? People choose a keto lifestyle, also known as a ketogenic lifestyle, for health reasons. The keto lifestyle has long been established as an effective treatment for childhood epilepsy. Many people choose the keto lifestyle for its effectiveness with weight management. Additionally, evidence suggests the keto lifestyle can be used to treat diabetes by providing people with diabetes who can't efficiently use glucose (carbohydrates) due to an inability to produce sufficient insulin or due to insulin resistance with an alternative fuel source to glucose, ketone bodies, which do not utilize insulin. Furthermore, there are many studies being conducted to determine the keto lifestyle's potential as a treatment for many other health conditions including cancers, Alzheimer's and Parkinson's Disease. The benefits of the ketogenic lifestyle result from maintaining a state of ketosis by consuming very low carbohydrate and very high fat foods and beverages which is the inspiration for the present keto beverages invention.

The Keto beverages in this present invention comprise by volume very low carbohydrate content, high fat content, low protein content and zero artificial sweeteners. These beverages are carbonated and contain a high fat content dairy and/or non-dairy cream (over 3 grams fat per tbsp). This present invention includes both alcoholic and non-alcoholic beverages. The alcoholic beverages comprise 5-15% alcohol by volume (ABV).

Keto alcoholic beverages such as in this present invention do not currently exist in the commercial marketplace. Alcoholic distilled-spirits cocktails currently available in the commercial market such as Margaritas, White Russians, The Cape Codder (vodka and cranberry juice), Screw Drivers (vodka and orange juice) and Bloody Marys as examples are not keto beverages because these traditionally formulated drinks contain too many grams of carbohydrates and/or too few grams of fat to promote ketosis. The currently available distilled-spirits mixed liquor drinks which contain under 3 grams of carbohydrates per 12 ounces, do not contain fat as the primary macro nutrient. High fat content is required to be an effective Keto Beverage. Conversely, the distilled-spirits liquor mixed drinks currently commercially available which contain significant fat content also have significant carbohydrate content—10+ grams of carbohydrates per 12 ounce serving. Additionally, there are many alcoholic beverages available in the commercial market called hard seltzers which contain carbonated water and a non-distilled-spirits alcohol. However, these drinks are not keto beverages because they contain 0 fat grams.

The distilled spirits alcoholic beverages in this invention have a range of 5-15% alcohol by volume (ABV), 0-3 grams of carbohydrates per 12 ounces, 10-25 grams of fat per 12 ounces and zero artificial sweeteners. No commercially available alcoholic beverages have that combination of characteristics. Furthermore, none of the commercially available alcoholic beverages have a combination of carbonated water and high fat cream which results not only in beverages with very low carbohydrate content, and high fat content but also creates alcoholic beverages with a unique creamy, carbonated flavor and mouth feel.

The non-alcoholic beverages currently available in the commercial market have similar keto beverage deficiencies with characteristics of too few grams of fat and/or too many grams of carbohydrates and/or artificial sweetener ingredient(s). Additionally, some non-alcoholic beverages also contain too much protein to be a Keto Beverage such as the category of drinks called protein shakes and kefirs. The Keto beverages in this invention contain 0 to less than 5 grams of protein. Furthermore. none of the commercially available non-alcoholic drinks have a combination of carbonated water and high fat cream which not only results in non-alcoholic beverages with very low carbohydrate content and high fat content but also creates non-alcoholic beverages with a unique creamy, carbonated flavor and mouth feel.

Thus, what is needed is a uniquely formulated Keto alcoholic beverage and a uniquely formulated Keto non-alcoholic beverage, which contains 0-3 grams of carbohydrates per 12 ounce serving, 10-25 grams of fat per 12 ounce serving, 0-5 grams of protein per 12 ounce serving and no artificial sweeteners, such as the present invention.

### SUMMARY OF THE INVENTION

The present invention relates to an alcoholic beverage and a non-alcoholic beverage composition intended for individuals on a Keto Diet comprising about 10-25 grams of fat, about 3 grams or less of carbohydrates, about 5 grams or less of protein, about 1-2 ounces of a high fat cream, about 5-11 ounces of carbonated water, about 5 grams or less of medium-chain triglycerides (MCT), and wherein the composition is per 12 ounce serving. The container and serving sizes of this present invention may vary with the ratios of the ingredients and the ratios of the macro nutrients remaining constant.

The present invention further relates to methods for producing a Keto beverage composition comprising the steps of mixing beverage formulation ingredients, carbonating the beverage formulation ingredients, and if required homogenizing and/or pasteurizing the beverage formulation ingredients, wherein said beverage formulation ingredients comprise about 3 grams or less of carbohydrates, about 10-25 grams of fat, about 5 grams or less of protein, about 5 grams or less of medium-chain triglycerides (MCT), about 250 milligrams or less of a carnitine, and about 5 grams or less of a creatine, and wherein the composition is per 12 ounce serving, and carbonating the beverage formulation ingredients comprises a pre-mix system or a post-mix system, where said pre-mix system combines first all of the beverage formulation ingredients and then carbonates the mixed beverage formulation; wherein the carbonation process involves either injecting carbon dioxide gas into a stream of water or adding carbon dioxide gas to a static liquid in a pressurized vessel, where said carbonated beverage formulation is then tunnel pasteurized and filled into containers, where said post-mix system combines all the ingredients except most of the liquids and creates a syrup which is 15-20% of the final volume wherein said syrup, prior to dilution, may be homogenized and/or pasteurized if required. The liquids are then separately chilled and carbonated; wherein the carbonation process involves either injecting carbon dioxide gas into a stream of water or adding carbon dioxide gas to a static liquid in a pressurized vessel; wherein said carbonated diluting liquid is combined in the required amounts to said syrup using a proportioning system to produce the complete carbonated formulation; where said formulation is then fed to a filler for packaging.

### BRIEF DESCRIPTION OF THE FIGURES

FIG. 1 shows the composition of an alcoholic keto beverage.

FIG. 2 shows the composition of a non-alcoholic keto beverage.

FIG. 3 shows a method for creating an alcoholic keto beverage.

FIG. 4 shows a method for creating a non-alcoholic keto Beverage.

### DETAILED DESCRIPTION OF THE INVENTION

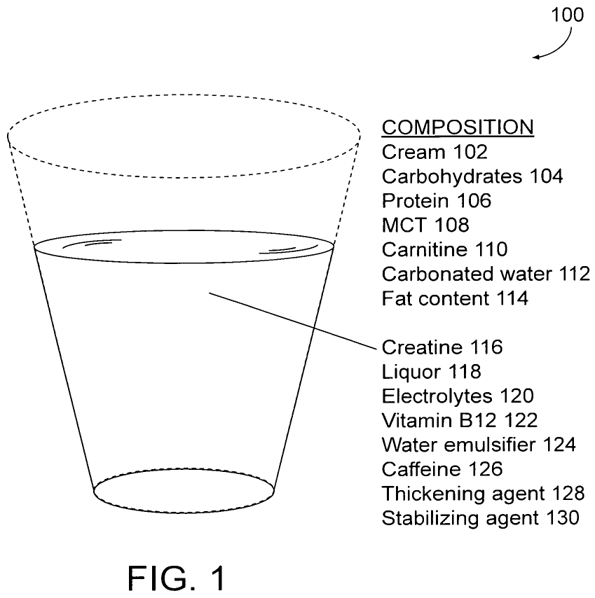

FIG. 1. depicts a 12-ounce container comprising a keto alcoholic beverage composition 100 having carbonated water 112, carbohydrates 104 102, carbohydrates 104, protein 106, medium-chain triglycerides (MCT) 108, carnitine 110, fat content 114, creatine 116, liquor/alcoholic 118, electrolytes 120, vitamin B12 122, water emulsifier 126, caffeine 128, thickening agent 130, and stabilizing agents 132.

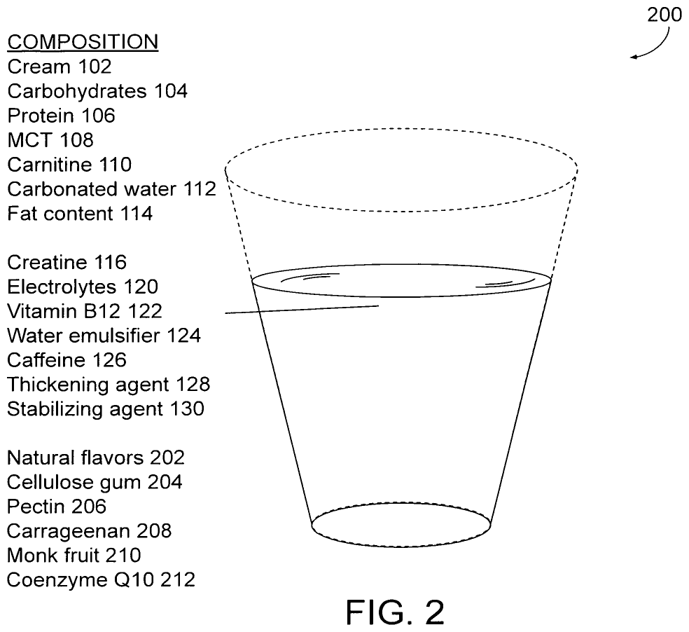

FIG. 2 depicts a 12 ounce container comprising a keto non-alcoholic beverage composition 200 having carbonated water 112, cream 102, carbohydrates 104, protein 106, medium-chain triglycerides (MCT) 108, carnitine 110, fat content 114, creatine 116, liquor/alcoholic 118, electrolytes 120, vitamin B12 122, water and cream emulsifier 126, caffeine 128, thickening agent 130, stabilizing agents 132, natural flavors 202, cellulose gum 204, pectin 206, carrageenan 208, monk fruit 210, coenzyme Q10 212.

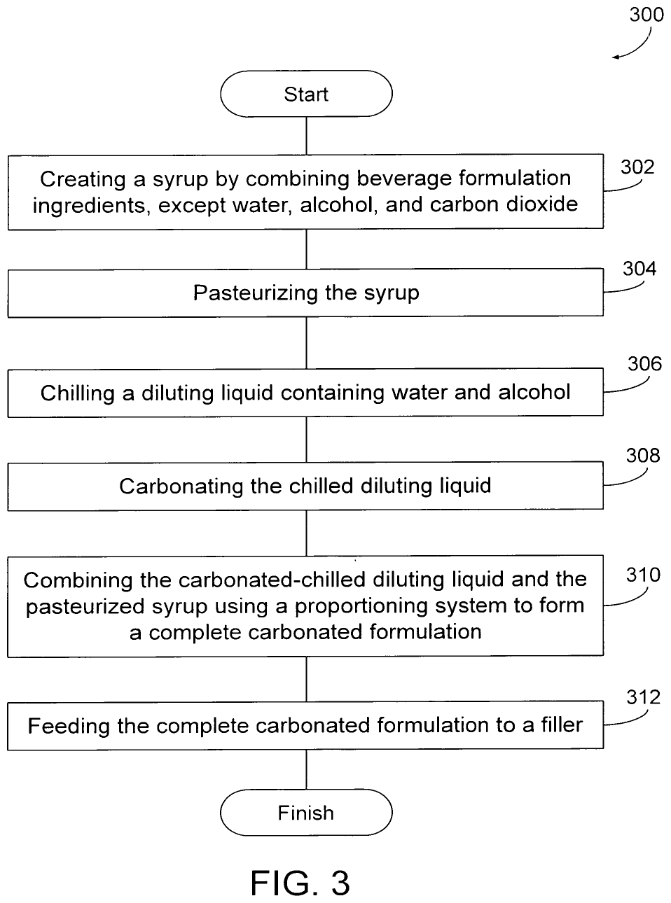

FIG. 3 shows a method for creating the alcoholic keto beverage 100 as a flowchart 300. Step one 302 comprise creating a syrup by combining beverage formulation ingredients, except water, distilled-spirits liquor/alcohol, and carbon dioxide. Step two 304 is pasteurizing the syrup. Step three 306 is chilling a diluting liquid containing water and alcohol. Step four 308 is carbonating the chilled diluting liquid. Step five 310 is combining the carbonated-chilled diluting liquid and the pasteurized syrup using a proportioning system to form a complete carbonated formulation. Step six 312 comprises feeding the complete carbonated formulation to a filler.

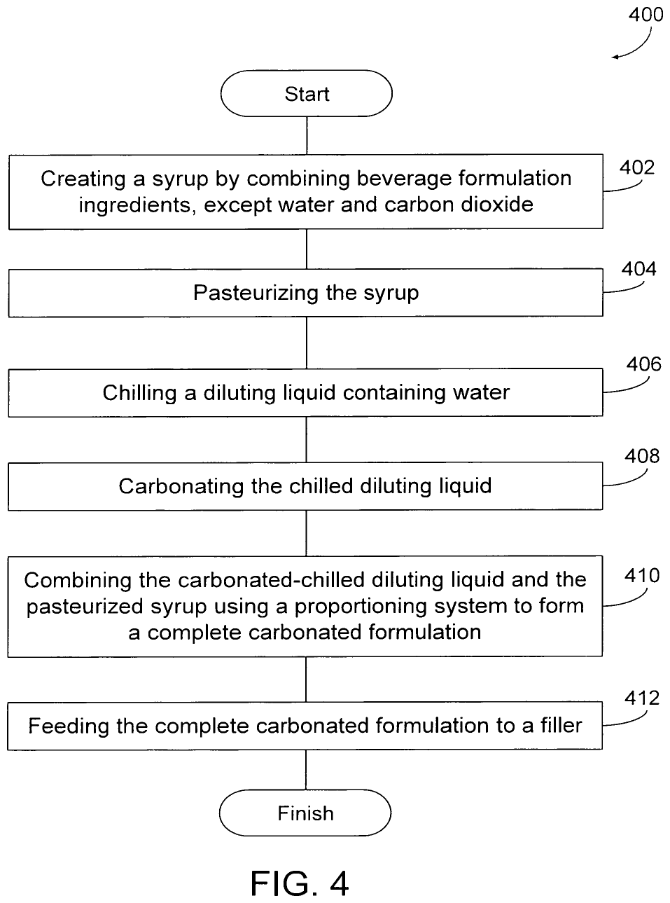

FIG. 4 shows a method for creating a non-alcoholic keto beverage 100 as a flowchart 400. Step one 402 comprises creating a syrup by combining beverage formulation ingredients, except water and carbon dioxide. Step two 404 is pasteurizing the syrup. Step three 406 is chilling a diluting liquid comprising water. Step four 408 is carbonating the chilled diluting liquid. Step five 410 is combining the carbonated-chilled diluting liquid and the pasteurized syrup using a proportioning system to form a complete carbonated formulation. Step six 412 is feeding the complete carbonated formulation to a filler.

In a preferred embodiment, these keto beverages comprise a macronutrient content of 75-100% fat content 114, 10-25 grams of fat, less than 3 grams of carbohydrates 104 and less than 5 grams of protein 106 per 12 oz serving per volume.

In another embodiment, the keto beverage composition comprises, 0 grams of protein 106 per 12 oz serving per volume.

In a preferred embodiment, the keto beverage composition comprises 0 grams of carbohydrates 104 per 12 oz serving per volume.

In a preferred embodiment, the keto beverage composition comprises 20 grams or less of fat per 12 oz serving per volume.

In another preferred embodiment, the keto beverage composition comprises 15 grams or less of fat per 12 oz serving per volume.

In a preferred embodiment, the keto beverage composition comprises 10 grams or more of fat per 12 oz serving per volume.

In another preferred embodiment, the non-alcoholic Keto Beverage composition comprises 1-2 ounces of cream 102 and 9-11 ounces of carbonated water 112 per 12 oz serving per volume.

In a preferred embodiment, the Keto alcoholic beverage composition comprises, per 12 ounce serving per volume, 1-2 ounces of cream 102, 5-9.5 ounces carbonated water 112 and 1.5-4.5 ounces alcohol which equates to 5%-15% ABV (Alcohol by Volume) using, but not limited to, 80 proof alcohol.

In a preferred embodiment, the cream 102 is, but not limited to, dairy heavy whipping cream, dairy cream, or coconut cream., or a high fat coconut milk cream.

In another embodiment, all of the beverage formulation ingredients except the carbonation are mixed, wherein said mix is carbonated and then tunnel pasteurized.

In one embodiment, the cream 102 being used, but not limited to, is a dairy heavy whipping cream or a dairy heavy cream or a coconut cream, comprising 15 or more grams of fat.

In another embodiment, the cream 102 being used comprises up to 20 grams of fat.

In one embodiment, the cream 102 being used comprises 10 or more grams of fat.

In a preferred embodiment, the distilled-spirit liquor used is, but not limited to, rum, vodka, whiskey or tequila,

In a preferred embodiment, per 12 oz serving by volume, the Keto alcoholic and non-alcoholic beverage compositions comprise less than 5 grams of protein 106.

In a preferred embodiment, each Keto alcoholic beverage composition comprises 5-15% alcohol by volume (ABV).

An example of electrolytes 120 being used in the present invention, but not limited to, is sodium, potassium, magnesium or calcium.

In another embodiment, the alcoholic or non-alcoholic beverage composition comprises less than 3 grams of carbohydrates 104.

In a preferred embodiment, the alcoholic or non-alcoholic beverage composition comprises 0 grams of carbohydrates 104.

In another embodiment, the alcoholic or non-alcoholic beverage composition comprises-less than 4 grams of protein 106.

In another embodiment, the alcoholic or non-alcoholic beverage composition further comprises coenzyme Q10 212 (Ubidecarenone), which has cardiovascular strengthening properties enabling higher levels of ketogenic energy production.

In another embodiment, the alcoholic or non-alcoholic beverage composition further comprises vitamin B12 122 which is a critical component for red blood cell production.

In another embodiment, the present invention, comprises low carbohydrate 104 and low protein 106 emulsifying, thickening and stabilizing agents. Many of these critical ingredients that are commercially available contain carbohydrates 104 and/or protein 106. In the present invention, carefully selected 0 or low carbohydrate and protein 106 emulsifying, thickening and stabilizing agents include, but are not limited to, guar gum for thickening and stabilizing, carrageenan 208 for thickening and stabilizing, glucomannan for thickening and emulsifying, mono and diglycerides for emulsifying, polysorbate 80 for stabilizing and emulsifying, and/or pectin 206 for thickening and stabilizing and/or other hydrocolloids for stabilizing.

In one embodiment, the beverage composition further comprises a cream 102 and a water emulsifier 126, such as, but not limited to, lecithin.

In another embodiment, the beverage composition further comprises a stabilizer for a cream 102, water and alcohol beverage, such as, but not limited to, sodium citrate.

In another embodiment, the beverages further comprise carnitine 110, a quaternary ammonium compound, which promotes energy metabolism by transporting long-chain fatty acids into mitochondria to be oxidized for energy production.

In another embodiment, the beverage composition further comprises a high fat content coconut milk.

In another embodiment, the alcoholic or non-alcoholic beverage further comprises natural flavors 202, cellulose gum 204 (sodium carboxymethylcellulose-CMC) and/or pectin 206 and/or carrageenan 208 for texture, flavor and as a thickening agent 130 and/or other hydrocolloid stabilizers.

In a preferred embodiment, the alcoholic or non-alcoholic beverage comprises additional ingredients for health benefits including electrolytes 120 to promote the health of multiple organ systems and in another embodiment, further comprises creatine 116, a high intensity exercise boosting supplement, and/or caffeine 128 as a source of energy.

In preferred embodiments, the alcoholic beverages comprise, but not limited to, either vodka, rum, tequila or whiskey.

The high fat dairy heavy whipping cream/heavy cream, in addition to adding fat, texture and flavor, can prevent curdling in the alcoholic versions of these unique Keto Beverages. The lower the fat of a milk or cream 102 contained in an alcoholic beverage, the more likely the drink will curdle. The heavy cream/heavy whipping cream helps to prevent curdling and maintain the emulsification of the beverages.

Examples of some formulations, but not limited to, based on 12 oz serving per volume, are listed below:
Creamy Coffee
Concentrated coffee, 10.5 oz carbonated water 112, 1.5 oz dairy cream/heavy cream/ heavy whipping cream.
Alcoholic Version comprises Concentrated coffee, 8 oz carbonated water 112, 1.5 oz dairy cream/heavy cream/heavy whipping cream, 2.5 oz vodka or rum which is 8% alcohol by volume (ABV) and natural flavors 202.
Creamy Lime
10 oz carbonated water 112, 1.5 oz dairy cream/heavy cream/heavy whipping cream, ½ oz lime juice and natural flavors 202.
Alcoholic version comprises 7.5 oz carbonated water 112, 1.5 oz heavy cream/heavy whipping cream, ½ oz lime juice and 2.5 oz tequila, vodka or rum which is 8% alcohol by volume (ABV) and natural flavors 202.
Creamy Orange
10.5 oz carbonated water 112, 1.5 oz dairy cream/heavy cream/heavy whipping cream and natural flavors 202.
Alcoholic version comprises 8 oz carbonated water 112, 1.5 oz heavy cream/heavy whipping cream and 2.5 oz Vodka or Rum which is 8% alcohol by volume (ABV) and natural flavors 202.
Creamy Strawberry
10.5 oz carbonated water 112, 1.5 oz dairy cream/heavy cream/heavy whipping cream, ca and natural flavors 202.
Alcoholic version comprises 8 oz carbonated water 112, 1.5 oz dairy cream/heavy cream/heavy whipping cream and 2.5 oz Vodka or Rum which is 8% alcohol by volume (ABV) and natural flavors 202
Creamy Pineapple Coconut
7.5 oz carbonated water 112, 1.5 oz dairy cream/heavy cream/heavy whipping cream, 3 oz unsweetened coconut milk and natural flavors 202.
In another embodiment, the alcoholic version comprises 6 oz carbonated water 112, 1.5 oz dairy heavy cream/heavy whipping cream, 2 oz unsweetened coconut milk, 2.5 oz Vodka or Rum which is 8% alcohol by volume (ABV) and natural flavors 202
Though the above formulations are based on 12 ounce servings, the container and serving sizes of this present invention may vary. For additional serving sizes, the ratios of each formulation's ingredients will remain constant and the ratios of each formulation's macro nutrients will remain constant, comprising high fat content, very low carbohydrate content and low protein content.

In order to produce ketones, the diet must contain a very high amount of fat, very low amount of carbohydrates 104—under 30 grams (1 cup serving of rice contains about 45 grams of carbohydrates) and restricted protein but not as restricted as carbohydrates 104—under 80 grams (a 5 oz steak has about 43 grams of protein). Therefore, to promote the production of ketones, the beverages formulated in this present invention are high in fat, 10-25 grams, very low in carbohydrates 104, 3 grams or less, and low in protein, 106. 5 grams or less. There is no calorie restriction on the Keto Diet, however, since people who are not strictly adhering to a Keto Lifestyle may consume these beverages, each 12-ounce serving of the alcoholic Keto Beverage in this present invention will not exceed 350 calories and each 12-ounce serving of the non-alcoholic Keto Beverage in this present invention will not exceed 225 calories.

These beverages are both non-alcoholic and alcoholic with up to 15% alcohol by volume (ABV) in the alcoholic formulations as a preferred embodiment.

In a preferred embodiment, per serving, the Keto alcoholic and non-alcoholic beverages comprise less than 3 grams carbohydrates 104, 0 to less than 5 grams protein 106 and 15 plus grams of fat.

In a preferred embodiment, the beverages comprise carbonated water 112, 0 carbohydrate heavy cream or heavy whipping cream and 0 carbohydrate, 0 protein 106 or extremely low carbohydrate and low protein stabilizers, emulsifiers and thickeners.

In a preferred embodiment, the beverages comprise additional ingredients for health benefits including electrolytes 120 to promote the health of multiple organ systems; carnitine 110, a quaternary ammonium compound, which promotes energy metabolism by transporting long-chain fatty acids into mitochondria to be oxidized for energy production and creatine 116, a high intensity exercise boosting supplement.

### Beverage Formulation Processing Methods

Process for Producing and Bottling these Keto Beverage Formulations with Shelf Stability

There are 5 required steps for producing these keto beverage formulations. The sequence of the steps vary depending on the specific beverage processing options being used as there are multiple processing methods available for these formulations. The process options selected may be based in part on the impact to shelf stability, processing costs, availability of bottlers with specific processing capabilities and impact on taste. No matter what the order of the steps these 5 steps are required:
1. Mix the beverage formulation ingredients
Ensure an optimal pH range for shelf stability of 3.5-4.5
2. Carbonate the beverage formulation.
This carbonation process can be done using a Pre-Mix system in which all ingredients are mixed and then carbonated or using a Post Mix system in which a syrup without the diluting liquid is carbonated and then the remaining liquid in the formulation is added and all the beverage ingredients are mixed. The process of beverage carbonation involves either injecting the gas into a stream of water or adding it to the static liquid in a pressurized vessel. In addition to these mechanical carbonation methods, in another embodiment, a natural microbiology carbonation method is used using a yeast fermentation process to create carbonation.
3. Eliminate pathogens. For beverages containing dairy, pasteurization is often used to eliminate pathogens to ensure consumption safety. There are a few types of pasteurization methods which may be used all of which have pros and cons regarding availability of bottlers, costs, resulting product shelf stability and taste. The two most common pasteurization processes which could be used, but not limited to, to pasteurize these keto beverages are tunnel pasteurization and flash pasteurization such as High Temperature/Short Time (HTST) flash pasteurization. The optimal pasteurization process may be tunnel pasteurization due to its effectiveness in pasteurizing carbonated beverages. Pasteurization may not be required if an alternative process to pasteurization or added ingredients eliminate the pathogens to ensure consumer safety.
4. Fill and Seal Containers
The product must be filled and sealed in containers which will preserve the beverage and in containers which will not be degraded by carbonation. Glass, aluminum cans and plastic bottles such as polyethylene terephthalate bottles (PET) may be used as containers.
5. Place Beverage Labels on the Individual Containers
Labels shall include the Nutrition Facts and Ingredients. Stamp the individual containers with a Use by Date.
   Hybrid Process for Producing and Bottling these Keto Beverage Formulations
   This production method contains two distinct stages of processing.

### STAGE ONE: Production of Shelf-Stable Partial Formulation Beverages

Step 1: Combine all of the ingredients except the dairy cream/heavy cream/heavy whipping cream.
Step 2: Add carbonation to the partial formulation.
Step 3: Fill and seal into bulk plastic bottles such as polyethylene terephthalate bottles (PET).
Step 4: Label the bottles with nutrition facts, ingredients and use by date for the partial formulation.
This partial beverage formulation will be shelf-stable for many months.
STAGE TWO: Add the Dairy Cream and/or Non-Dairy Cream/Heavy Cream/Heavy Whipping Cream to the Shelf-Stable Partial Formulation Beverages
Completing this stage two process will result in the finished Keto Beverage formulation. This stage two process should be completed no more than 7 days prior to the beverage being consumed.
Step 1: Open a bottle which contains the shelf-stable partial formulations. Prior to opening ensure the formulation is mixed.
Step 2: Pour into individual serving bottles which are labeled with the nutrition facts and ingredients.
Step 3: Add the dairy cream and/or non-dairy cream/heavy Cream/heavy whipping cream to the individual serving bottles. The amount added is dependent on the specific formulation and the total ounces of the individual bottles-a range of 1-2 ounces dairy heavy cream/heavy whipping cream should be added per 12 oz serving. The dairy heavy cream/heavy whipping cream/cream being used must have a use by date that is at least 7 days in the future.
Step 4: Stamp a used by date on the individual bottles containing the complete Keto Beverage formulations.
Step 5: Cap and seal the individual bottles to promote freshness
Step 6: Keep these completed keto beverages refrigerated until the time of consumption.
Step 7: Shake gently and serve
This two-stage beverage production process is produced for quick consumption in such venues as bars, restaurants, food trucks/trailers at festivals and/or farmer's markets.
The beverage containers could be a glass or plastic bottle. If the beverage is filled in a plastic bottle, it may be a polyethylene terephthalate (PET) type of bottle to help keep the beverage stable.

While the foregoing written description of the invention enables one of ordinary skill to make and use what is considered presently to be, the best mode thereof, those of ordinary skill will understand and appreciate the existence of variations, combinations, and equivalents of the specific embodiment, method, and examples, herein.

What is claimed is:

1. A beverage composition comprising
a. about 2 ounces or less of a cream,
b. about 3 grams or less of carbohydrates,
c. about 5 grams or less of protein,
d. about 5 grams or less of medium-chain triglycerides (MCT),
e. About 250 milligrams or less of a carnitine,
f. up to 11 ounces of carbonated water.
g. wherein the composition contains about between 10-25 grams of fat, and
h. wherein the composition is per 12 oz serving.

2. The beverage composition of claim 1, further comprising up to 5 grams of a creatine.

3. The beverage composition of claim 2, further comprising a distilled-spirit liquor, wherein said liquor is about 1.5 to 4.5 ounces of alcohol equating to 5% to 15% alcohol by volume.

4. The beverage composition of claim 3, wherein said liquor is rum, vodka, tequila, or whiskey.

5. The beverage composition of claim 1, wherein the protein is about 0 grams and carbohydrate content is about 0 grams.

6. The beverage composition of claim 1, wherein the carbonated water is about 5 ounces to about 9.5 ounces.

7. The beverage composition of claim 1, further comprising electrolytes; vitamin B12; a cream and water emulsifier; caffeine; thickening and stabilizing agents; wherein said cream is a dairy heavy whipping cream, dairy cream, coconut cream, other high fat cream or a high fat coconut milk.

8. The beverage composition of claim 1, wherein protein is about 0 grams and carbohydrates are about 0 grams.

9. The beverage composition of claim 1, wherein the carbonated water is about 9 ounces to about 11 ounces.

10. The beverage composition of claim 1, further comprising electrolytes; vitamin B12; a cream and a water emulsifier; caffeine; coenzyme Q10; thickening and stabilizing agents; wherein said cream is dairy heavy whipping cream, cream, coconut cream, or a high fat coconut milk.

11. The beverage composition of claim 1, further comprising natural flavors and cellulose gum (sodium carboxymethylcellulose-CMC).

12. The beverage composition of claim 1, further comprising a pectin and/or a carrageenan.

13. The beverage composition of claim 1, further comprising monk fruit.

14. A method for producing a Keto beverage composition comprising the steps of:
a. mixing beverage formulation ingredients,
b. carbonating the beverage formulation ingredients, and
c. pasteurizing and/or homogenizing the beverage formulation ingredients, wherein said beverage formulation ingredients comprises about 3 grams or less of carbohydrates, about 5 grams or less of protein, about 5 grams or less of medium-chain triglycerides (MCT), about 250 milligrams or less of a carnitine, and about 5 grams or less of a creatine, wherein the composition contains about 10-25 grams of fat, and wherein the composition is per 12 oz serving.

15. The method of claim 14, wherein said beverage formulation ingredients further comprise electrolytes; vitamin B12; a cream and water emulsifier, caffeine, thickening and stabilizing agents.

16. The method of claim 14, further comprising a distilled-spirit liquor, wherein said liquor is about 1.5 to 4.5 ounces of alcohol equating to 5% to 15% alcohol by volume.

17. The method of claim 14, carbonating the beverage formulation ingredients comprises a pre-mix system, where said pre-mix system combines first the beverage formulation ingredients and then carbonates the mixed beverage formulation; wherein the carbonation process involves either a mechanical method or a natural method. The mechanical carbonation is done by either injecting carbon dioxide gas into a stream of water or adding carbon dioxide gas to a static liquid in a pressurized vessel. The natural carbonation process uses a microbe such as yeast to produce carbon dioxide through fermentation.

18. The method of claim 14, further comprising adding a dairy heavy whipping cream.

19. The method of claim 14, wherein carbonating the beverage formulation ingredients comprises a post-mix system, whereby all required ingredients except water, alcohol and carbon dioxide are combined to make a syrup, wherein said syrup is about 15-20% of the final volume, wherein said syrup, prior to dilution, may be-homogenized and/or pasteurized through a flash pasteurization process; wherein said post-mix system then separately chills a diluting liquid containing water and alcohol, and then carbonates the chilled diluting liquid; wherein the carbonation process involves either injecting carbon dioxide gas into a stream of water or adding carbon dioxide gas to a static liquid in a pressurized vessel; wherein said carbonated diluting liquid is combined in the required amounts to said syrup using a proportioning system to produce the complete carbonated formulation; where said formulation is then fed to a filler for packaging.

20. The method of claim 14, wherein said beverage formulation ingredients comprises about 10-25 grams of fat, about 0 grams of carbohydrates, and about 0 grams of protein.

21. The method of claim 14, wherein carbonating the beverage formulation ingredients comprises a post-mix system, whereby all required ingredients except water and carbon dioxide are combined to make a syrup, wherein said syrup, prior to dilution, may be homogenized or pasteurized through a flash pasteurization process; wherein said post-mix system then separately chills a diluting liquid comprising only water for a non-alcoholic beverage, and then carbonates the chilled diluting liquid; wherein the carbonation process involves either injecting carbon dioxide gas into a stream of water or adding carbon dioxide gas to a static liquid in a pressurized vessel; wherein said carbonated diluting liquid is combined in the required amounts to said syrup using a proportioning system to produce the complete carbonated formulation; where said formulation is then fed to a filler for packaging.

22. The beverage composition of claim 1, further comprising coenzyme Q10.
